(19) United States
(12) Patent Application Publication
(10) Pub. No.: US 2018/0002310 A1
(43) Pub. Date: Jan. 4, 2018

(54) CRYSTALLINE FORMS OF EFINACONAZOLE

(71) Applicant: Perrigo API Ltd., Bnei Brak (IL)

(72) Inventors: Itai ADIN, Beer-Sheva (IL); Hila ELAZARI-SHALOM, Beer-Sheva (IL); Ori RORILK, Beer-Sheva (IL); Yana SERY, Beer Sheva (IL)

(73) Assignee: Perrigo API Ltd., Bnei Brak (IL)

(21) Appl. No.: 15/544,173

(22) PCT Filed: Dec. 2, 2015
(86) PCT No.: PCT/IL15/51171

§ 371 Date: Jul. 17, 2017

Related U.S. Application Data
(60) Provisional application No. 62/105,390, filed on Jan. 20, 2015.

(51) Int. Cl. C07D 401/06

(52) U.S. Cl. CPC C07D 401/06; C07B 2200/13

(57) ABSTRACT

The present invention provides crystalline forms of efinaconazole, including crystalline efinaconazole forms designated herein as Form A, Form B and Form C, crystalline efinaconazole p-toluenesulfonate salt designated herein as Form I, and processes for their preparation.

### TECHNICAL FIELD

The invention relates to polymorphic forms of efinaconzole and processes for their preparation.

### BACKGROUND OF THE INVENTION

Efinaconazole, (2R,3R)-2-(2,4-difluorophenyl)-3-(4-methylenepiperidin-1-yl)-1-(1H-1,2,4-triazol-1-yl)butan-2-ol, has the following chemical structure:

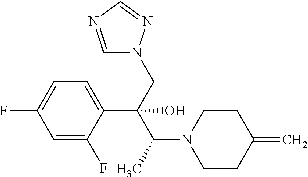 [SMILES: C=C1CCN([C@H](C)[C@](O)(Cn2cncn2)c2ccc(F)cc2F)CC1]

It is used as an antifungal, and marketed as the active pharmaceutical ingredient of a topical solution indicated for the treatment of onychomycosis.

Efinaconazole and a process for its preparation, as well as the p-toluenesulfonate salt of efinaconazole, were described in U.S. Pat. No. 5,620,994. The preparation of efinaconazole was later described in U.S. Pat. No. 8,871,942. Polymorphs of efinaconazole or its p-toluenesulfonate salt were not described.

The occurrence of different crystal structures of a solid material is known as polymorphism. A single molecule, such as efinaconazole, may give rise to various polymorphs having distinct crystal structures and physical properties. Different crystalline forms of the same molecule may differ, for example, with respect to their X-ray powder diffraction patterns, Raman fingerprints, and thermal behavior (as may be measured by differential scanning calorimetry or thermogravimetric analysis).

### SUMMARY OF THE INVENTION

The present invention provides crystalline forms of efinaconazole and processes for their preparation. Specifically, the invention provides crystalline efinaconazole forms designated herein as Form A, Form B and Form C.

The invention further provides crystalline efinaconazole p-toluenesulfonate salt, designated herein as Form I, and a process for its preparation.

### BRIEF DESCRIPTION OF THE DRAWINGS

FIG. 1 depicts the X-ray diffraction pattern of efinaconazole Form A.

FIG. 2 depicts the Raman spectrum of efinaconazole Form A.

FIG. 3 depicts the DSC thermogram of efinaconazole Form A.

FIG. 4 depicts the TGA thermogram of efinaconazole Form A.

FIG. 5 depicts the X-ray diffraction pattern of efinaconazole Form B.

FIG. 6 depicts the Raman spectrum of efinaconazole Form B.

FIG. 7 depicts the DSC thermogram of efinaconazole Form B.

FIG. 8 depicts the TGA thermogram of efinaconazole Form B.

FIG. 9 depicts the X-ray diffraction pattern of efinaconazole Form C.

FIG. 10 depicts the Raman spectrum of efinaconazole Form C.

FIG. 11 depicts the DSC thermogram of efinaconazole Form C.

FIG. 12 depicts the TGA thermogram of efinaconazole Form C.

FIG. 13 depicts the X-ray diffraction pattern of efinaconazole p-toluenesulfonate Form I.

### DETAILED DESCRIPTION OF THE INVENTION

In a first embodiment, the present invention provides crystalline efinaconazole designated herein as Form A. Crystalline Form A of efinaconazole is characterized by an X-ray powder diffraction pattern having peaks at at least two, preferably at least three, and more preferably all, of 7.6, 10.4, 10.8 and 24.0 degrees 2-theta±0.1 degrees 2-theta.

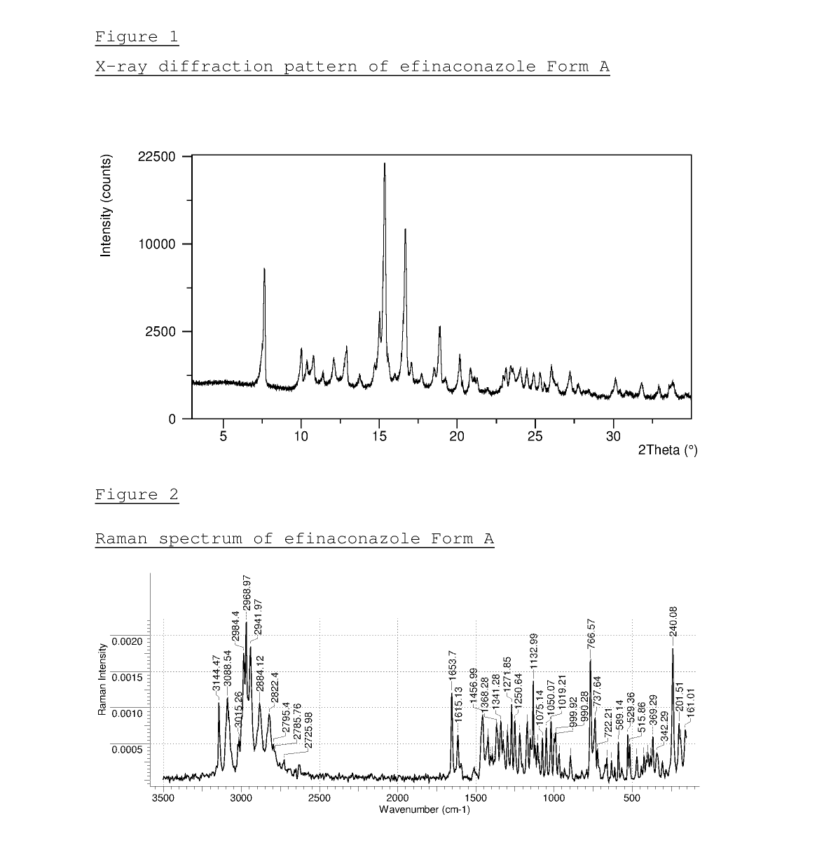

In a particular embodiment, efinaconazole Form A is characterized by an X-ray powder diffraction pattern substantially as depicted in FIG. 1. The X-ray powder diffraction peak positions (indicated as degrees 2-theta) and relative intensities (indicated as I/Id exhibited by efinaconazole Form A, as depicted in FIG. 1, are as follows (relative intensities are indicated in parentheses for each peak position): 7.6 (0.33), 10.0 (0.05), 10.4 (0.04), 10.8 (0.04), 11.4 (0.02), 12.1 (0.04), 12.9 (0.06), 13.7 (0.02), 15.0 (0.11), 15.3 (1.0), 16.7 (0.53), 17.1 (0.03), 17.7 (0.02), 18.5 (0.02), 18.9 (0.12), 19.3 (0.01), 20.2 (0.05), 20.8 (0.03), 23.1 (0.02), 23.4 (0.03), 24.0 (0.03), 24.5 (0.02), 24.9 (0.02), 25.3 (0.02), 26.0 (0.03), 27.2 (0.02), 27.8 (0.01), 30.1 (0.02), 31.8 (0.01), 32.9 (0.01) and 33.8 (0.01) degrees 2-theta±0.1 degrees 2-theta. Preferably, efinaconazole Form A is characterized by an X-ray powder diffraction pattern having peaks at at least two, preferably at least four, six or eight, and more preferably all, of the above degrees 2-theta values.

Crystalline efinaconazole Form A is further characterized by a Raman spectrum substantially as depicted in FIG. 2.

Crystalline efinaconazole Form A is further characterized by a DSC thermogram substantially as depicted in FIG. 3, and a DSC melting onset at about 85.75° C.±1.00° C.

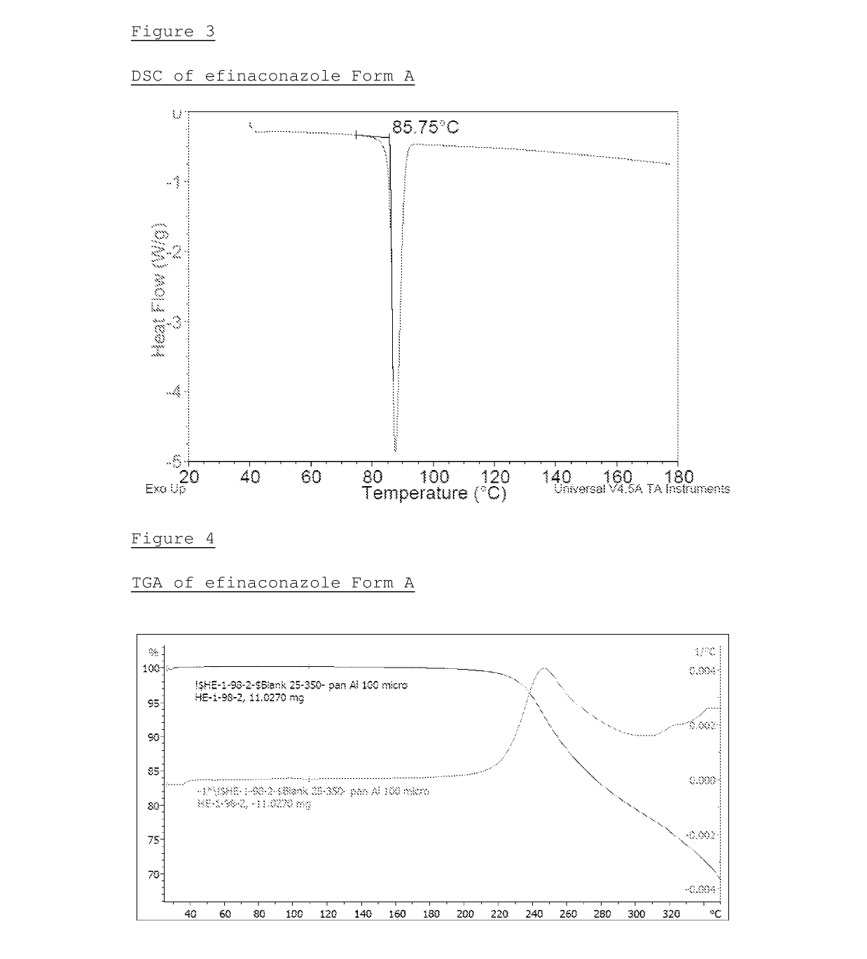

Crystalline efinaconazole Form A is further characterized by a TGA thermogram substantially as depicted in FIG. 4.

In addition, the present invention provides a process for the preparation of crystalline efinaconazole Form A, comprising the steps of dissolving efinaconazole in a suitable solvent, such as acetonitrile; adding a suitable anti-solvent, such as a mixture of water and ethanol in a ratio of 2:1; maintaining the mixture for a duration sufficient to allow the formation of crystals; and optionally isolating the obtained crystals.

The efinaconazole used as starting material can be prepared, for example, according to the procedures described in U.S. Pat. No. 5,620,994 or U.S. Pat. No. 8,871,942, both of which are incorporated herein by reference in their entirety.

The volume ratio between the solvent and the anti-solvent may be e.g. between about 1:10 and 10:1, and suitably about 1:3. Preferably, after the addition of the anti-solvent, the mixture is maintained for a duration of about 2 to 48 hours before isolation of the obtained crystals, and typically about 24 hours. The obtained crystals may be isolated from the reaction mixture by conventional means such as filtration.

As an example, efinaconazole Form A can be prepared by dissolving efinaconazole in acetonitrile, adding a 2:1 solution of water and ethanol, maintaining the mixture overnight at room temperature and collecting the obtained crystals by filtration.

In another embodiment, the invention provides crystalline efinaconazole designated herein as Form B. Crystalline Form B of efinaconazole is characterized by an X-ray powder diffraction pattern having peaks at at least two, preferably at least three, preferably at least four, and more preferably all, of 7.7, 10.0, 10.6, 12.5, 23.8 and 34.5 degrees 2-theta±0.1 degrees 2-theta.

In a particular embodiment, efinaconazole Form B is characterized by an X-ray powder diffraction pattern substantially as depicted in FIG. 5. The X-ray powder diffraction peak positions (degrees 2-theta) and relative intensities (I/Id exhibited by efinaconazole Form B, as depicted in FIG. 5, are as follows (relative intensities are indicated in parentheses for each peak position): 7.7 (0.37), 10.0 (0.05), 10.6 (0.12), 11.4 (0.01), 12.5 (0.14), 12.9 (0.03), 13.8 (0.01), 15.0 (0.18), 15.4 (1.00), 16.7 (0.68), 17.8 (0.01), 18.9 (0.26), 20.2 (0.06), 20.9 (0.02), 21.2 (0.04), 22.8 (0.01), 23.2 (0.02), 23.4 (0.04), 23.8 (0.06), 24.5 (0.05), 24.9 (0.02), 25.3 (0.03), 26.0 (0.06), 27.3 (0.04), 27.8 (0.01), 28.2 (0.02), 28.5 (0.01), 30.1 (0.02), 30.5 (0.01), 30.9 (0.01), 31.4 (0.01), 31.8 (0.01), 32.9 (0.01), 33.2 (0.01), 33.8 (0.02) and 34.5 (0.01) degrees 2-theta±0.1 degrees 2-theta. Preferably, efinaconazole Form B is characterized by an X-ray powder diffraction pattern having peaks at at least two, preferably at least four, six or eight, and more preferably all, of the above degrees 2-theta values.

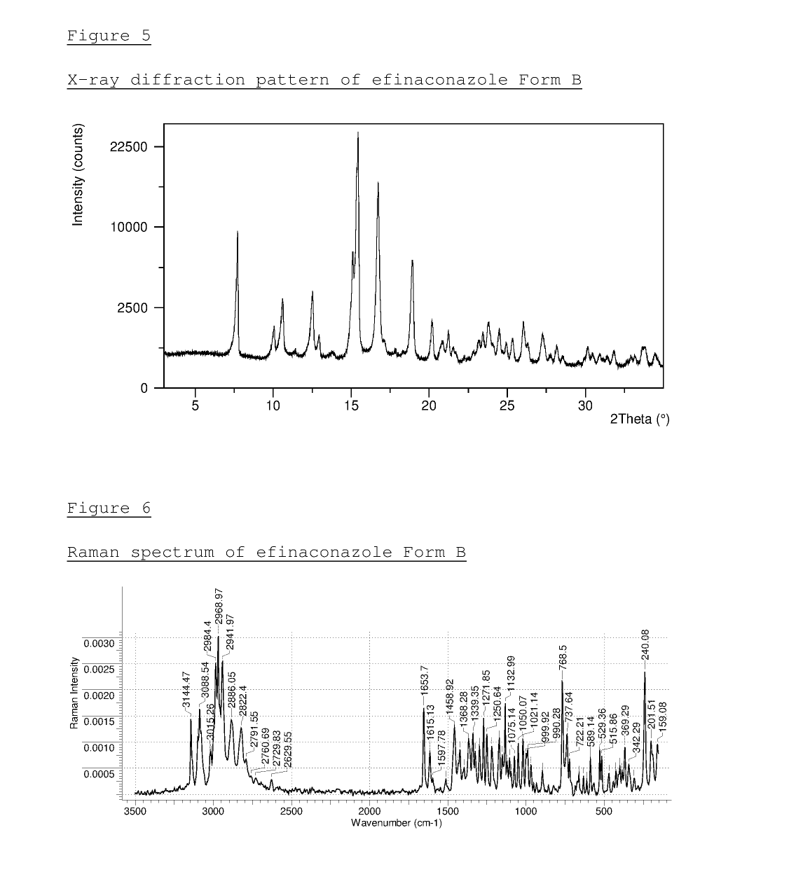

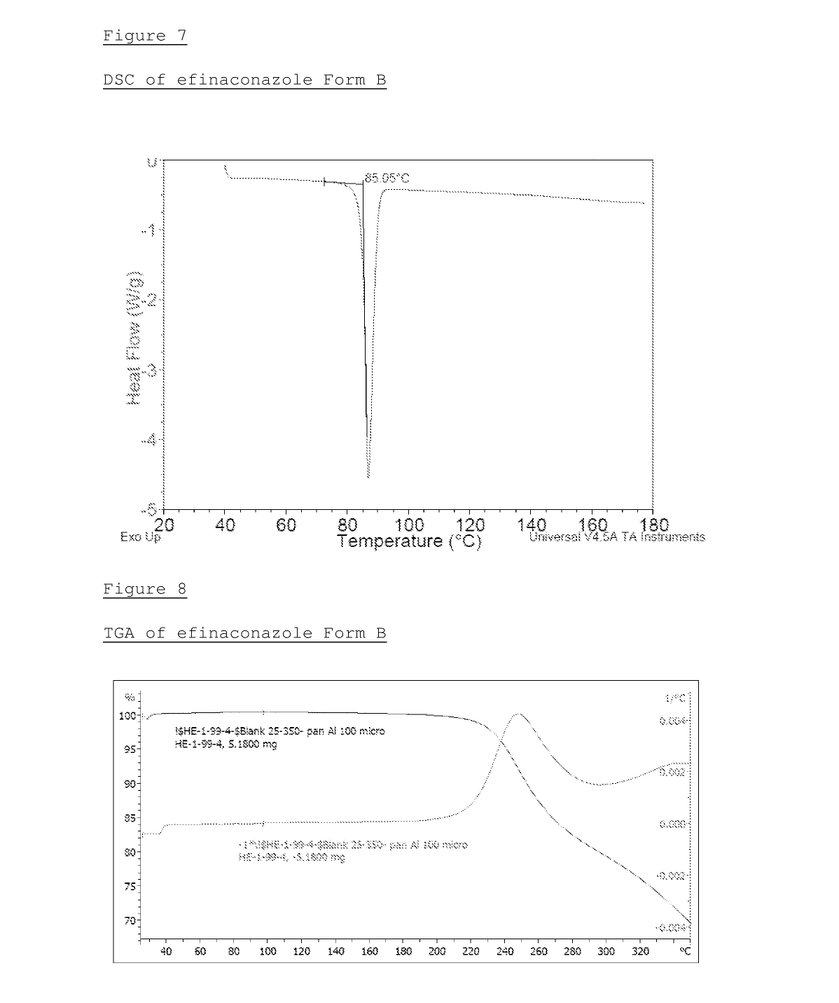

Crystalline efinaconazole Form B is further characterized by a Raman spectrum Raman spectrum substantially as depicted in FIG. 6.

Crystalline efinaconazole Form B is further characterized by a DSC thermogram substantially as depicted in FIG. 7, and a DSC melting onset at about 85.05° C.±1.00°.

Crystalline efinaconazole Form B is further characterized by a TGA thermogram substantially as depicted in FIG. 8.

In addition, the present invention provides a process for the preparation of crystalline efinaconazole Form B, comprising the steps of dissolving efinaconazole in a suitable solvent, such as diethylether or diisopropylether; adding a suitable anti-solvent, such as hexane; maintaining the mixture for a duration sufficient to allow the formation of crystals; and optionally isolating the obtained crystals.

The volume ratio between the solvent and the anti-solvent may be e.g. between about 1:10 and 10:1. In the case of using diethylether as the solvent and hexane as the anti-solvent, the ratio between the solvent and the anti-solvent may be e.g. between about 1:10 and 10:1, preferably between about 1:2 and 1:2.5, more preferably between about 1:2.1 and 1:2.2, with 1:2.13 being especially preferred. The obtained crystals may be isolated from the reaction mixture by conventional means such as filtration.

As an example, efinaconazole Form B can be prepared by dissolving efinaconazole in diethylether, adding hexane, maintaining the mixture overnight at room temperature and collecting the obtained crystals by filtration.

As another example, efinaconazole Form B can be prepared by dissolving efinaconazole in a 1:1 mixture of diisopropylether and hexane at 50° C., cooling to 5° C. over 1.5 hours, stirring at 5° C. for another 0.5 hour and collecting the obtained crystals by filtration.

Alternatively, crystalline efinaconazole Form B may be obtained by dissolving efinaconazole in hexane, preferably at an elevated temperature (such as between about 40° C.-70° C., and suitably about 60° C.), following which the solution is cooled to a lower temperature (such as, for example, room temperature). Optionally, hexane may be added. The obtained crystals may be isolated from the reaction mixture by conventional means such as filtration.

In another embodiment, the invention provides crystalline efinaconazole designated herein as Form C. Crystalline Form C of efinaconazole is characterized by an X-ray powder diffraction pattern having peaks at at least two, preferably at least three, preferably at least four, and more preferably all, of 7.7, 10.1, 11.4, 13.8, 13.9, 17.9, 22.5, 26.2, 26.4 and 29.2 degrees 2-theta±0.1 degrees 2-theta.

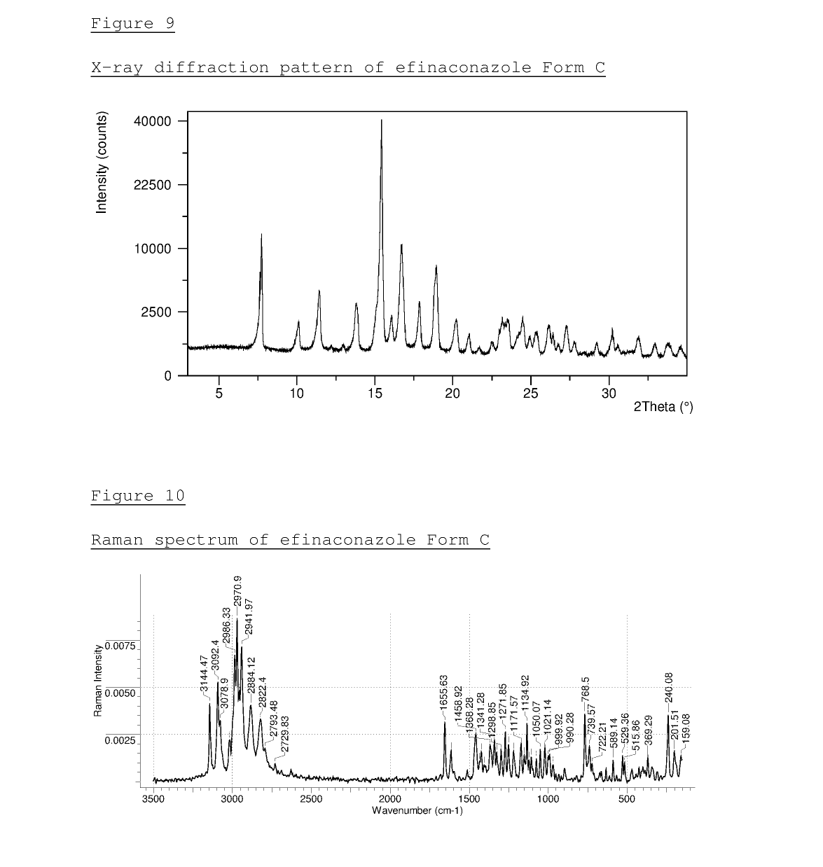

In a particular embodiment, efinaconazole Form C is characterized by an X-ray powder diffraction pattern substantially as depicted in FIG. 9. The X-ray powder diffraction peak positions (degrees 2-theta) and relative intensities (I/Id exhibited by efinaconazole Form C, as depicted in FIG. 9, are as follows (relative intensities are indicated in parentheses for each peak position): 7.7 (0.21), 10.1 (0.04), 11.4 (0.11), 13.0 (0.01), 13.9 (0.07), 15.4 (1.00), 16.1 (0.05), 16.7 (0.27), 17.9 (0.08), 19.0 (0.18), 20.2 (0.04), 21.0 (0.02), 22.5 (0.01), 23.2 (0.04), 23.6 (0.04), 24.5 (0.05), 24.9 (0.02), 25.4 (0.02), 26.2 (0.03), 26.8 (0.01) 27.3 (0.03), 27.8 (0.01), 29.2 (0.01), 30.2 (0.03), 30.6 (0.01), 31.8 (0.02), 32.9 (0.01), 33.7 (0.01), 33.9 (0.01), 34.6 (0.01) degrees 2-theta±0.1 degrees 2-theta. Preferably, efinaconazole Form C is characterized by an X-ray powder diffraction pattern having peaks at at least two, preferably at least four, six or eight, and more preferably all, of the above degrees 2-theta values.

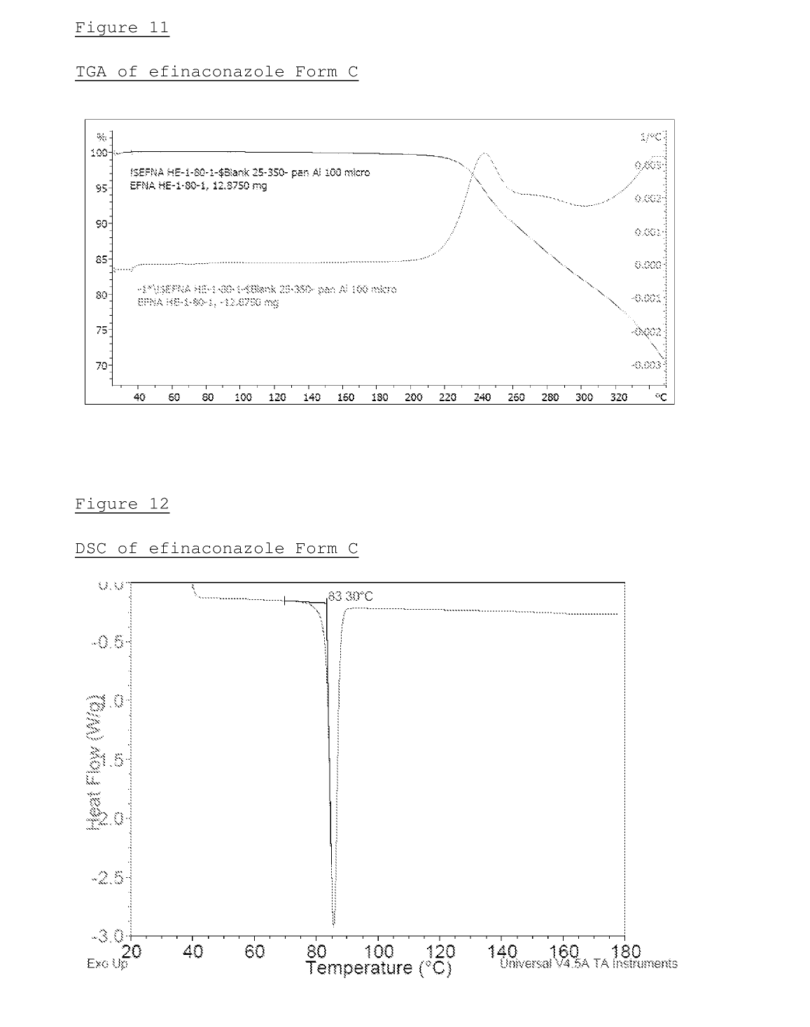

Crystalline efinaconazole Form C is further characterized by a Raman spectrum substantially as depicted in FIG. 10.

Crystalline efinaconazole Form C is further characterized by a DSC thermogram substantially as depicted in FIG. 11, and a DSC melting onset at about 83.30° C.±1.00° C.

Crystalline efinaconazole Form C is further characterized by a TGA thermogram substantially as depicted in FIG. 12.

In addition, the present invention provides a process for the preparation of crystalline efinaconazole Form C, comprising the steps of dissolving efinaconazole in a suitable solvent, such as a 1:1 mixture of diisopropylether and hexane; maintaining the mixture for a duration sufficient to allow the formation of crystals, and optionally isolating the obtained crystals.

The dissolution of efinaconazole in the solvent may be carried out at an elevated temperature, such as 40° C.

After complete dissolution of the efinaconazole, the mixture is preferably cooled to a temperature of about 4-5° C. The mixture is then preferably maintained for a duration of over 2 hours, such as, for example, between about 12 to 48 hours, and typically about 24 hours, before isolation of the obtained crystals. The crystals may be isolated from the reaction mixture by conventional means such as filtration.

As an example, efinaconazole Form C can be prepared by dissolving efinaconazole in a 1:1 mixture of diisopropylether and hexane at 40° C., cooling to 4-5° C., maintaining the mixture overnight and collecting the obtained crystals by filtration.

Alternatively, crystalline efinaconazole Form C may be obtained by dissolving efinaconazole in cyclohexane, preferably at an elevated temperature (such as between about 40° C.-70° C., and suitably about 55° C.), following which the solution is cooled to a lower temperature (such as between about 0° C.-25° C., and suitably about 10° C.). The obtained crystals may be isolated from the reaction mixture by conventional means such as filtration.

In another embodiment, the invention provides crystalline efinaconazole p-toluenesulfonate salt, designated herein as Form I. Form I is characterized by an X-ray powder diffraction pattern having peaks at at least two, preferably at least three, preferably at least four, and more preferably all, of 7.0, 9.2, 18.4, 20.9 and 22.9 degrees 2-theta±0.1 degrees 2-theta.

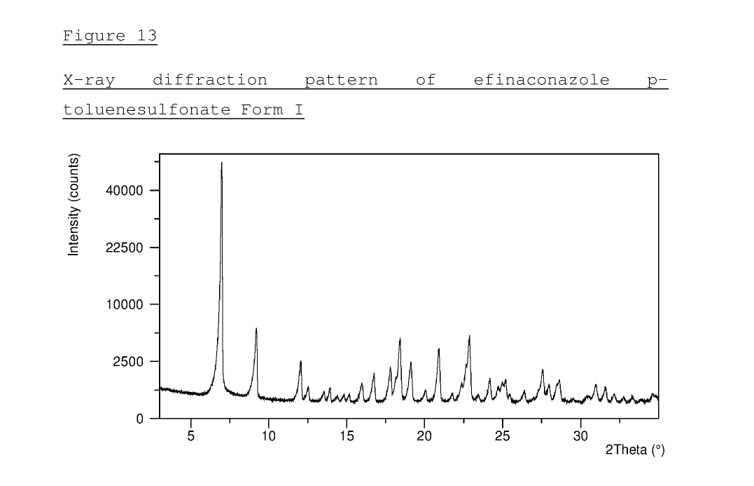

In a particular embodiment, Form I is characterized by an X-ray powder diffraction pattern substantially as depicted in FIG. 13. The X-ray powder diffraction peak positions (indicated as degrees 2-theta) and relative intensities (indicated as I/Id exhibited by efinaconazole Form I, as depicted in FIG. 13, are as follows (relative intensities are indicated in parentheses for each peak position): 7.0 (1.00), 9.2 (0.10), 12.0 (0.05), 12.5 (0.01), 13.5 (0.01), 13.9 (0.01), 16.0 (0.01), 16.7 (0.03), 17.8 (0.04), 18.4 (0.10), 19.1 (0.05), 20.1 (0.01), 20.9 (0.07), 22.9 (0.10), 24.2 (0.02), 24.7 (0.01), 25.1 (0.02), 26.4 (0.01), 27.6 (0.03), 28.0 (0.01), 28.7 (0.02), 31.0 (0.01) and 31.6 (0.01) degrees 2-theta±0.1 degrees 2-theta. Preferably, Form I is characterized by an X-ray powder diffraction pattern having peaks at at least two, preferably at least four, six or eight, and more preferably all, of the above degrees 2-theta values.

In addition, the present invention provides a process for the preparation of crystalline efinaconazole p-toluenesulfonate salt Form I, comprising the steps of dissolving efinaconazole p-toluenesulfonate in a suitable solvent, and subsequently causing the precipitation of efinaconazole p-toluenesulfonate by methods known in the art such as cooling the solution, evaporating the solvent or adding an anti-solvent. The obtained crystals may optionally be isolated.

The efinaconazole p-toluenesulfonate salt used as starting material can be prepared, for example, according to the procedure described in U.S. Pat. No. 5,620,994. Suitable solvents for dissolving the efinaconazole p-toluenesulfonate include, for example, methyltetrahydrofuran, tetrahydrofuran, acetone, acetonitrile or mixtures thereof. Preferably, the dissolution of efinaconazole p-toluenesulfonate is carried out at an elevated temperature, such as between about 40-70° C. In a preferred embodiment, after dissolution of the efinaconazole p-toluenesulfonate, crystals are allowed to form by cooling the solution temperature. Suitably, the solution is cooled to a temperature of, for example, between about 5-20° C. The solution may be maintained at that temperature for a duration of about 2 to 48 hours, and typically about 24 hours, before isolation of the obtained crystals. The obtained crystals may be isolated from the reaction mixture by conventional means such as filtration.

In an embodiment of the invention, the various crystalline forms of efinaconazole of the present invention are in substantially pure form. The term “substantially pure”, as used herein, refers to crystalline forms of, or greater than, 80%, preferably 90%, more preferably 95%, more preferably 96%, 97%, 98% or 99% polymorphic purity, as determined, for example, by X-ray powder diffraction or by Raman spectroscopy.

The crystalline forms of the invention can be used to prepare other solid state forms of efinaconazole or efinaconazole salts.

The crystalline forms of efinaconazole or its p-toluenesulfonate salt of the present invention can be used to prepare pharmaceutical compositions. Such compositions may include, for example, topical solutions for the treatment of onychomycosis. The pharmaceutical compositions can be prepared by methods known in the art, such as, for example, the methods described in U.S. Pat. No. 8,039,494, U.S. Pat. No. 8,486,978, WO2008081940 or WOWO2009085314, all of which are incorporated herein by reference in their entirety.

The invention thus provides the use of an efinaconazole polymorph selected from the group consisting of Form A, Form B and Form C, or a mixture thereof, or of efinaconazole p-toluenesulfonate Form I, in the preparation of a pharmaceutical composition. Further provided by the invention are pharmaceutical compositions prepared from an efinaconazole polymorph selected from the group consisting of Form A, Form B and Form C, or a mixture thereof, or from efinaconazole p-toluenesulfonate Form I.

The invention is further illustrated by the following examples, which are not to be construed as limiting.

### EXAMPLES

All percentages herein are weight percentages unless otherwise indicated.

Where used herein, the term “room temperature” refers to a temperature in the range from about 20° C. to 30° C., such as, for example, 25° C.

### Methods

X-ray diffraction was measured using Panalytical X-ray diffractometer model X'Pert Pro. System description: CuK-Alpha1 wavelength=1.54060, voltage 45 kV, current 30 mA, diversion slit=0.25°, anti-scattering slit=0.5°. Sample stage=Reflection-Transmission Spinner. Sample mode: Reflexion. Detector—X'Celerator.

### Measurement Parameters:

Start Position [° 2Th.]: 3; End Position [° 2Th.]: 35; Step Size [° 2Th.]: 0.004. Scan Step Time [s]: 10.

Differential scanning calorimetry (DSC) measurements were carried on TA instruments model Q1000. Purge gas: dry air (dew point −80° C.). Sample pans: crimped Aluminum.

Heating rate: 10° C./min.

Thermogravic analysis (TGA) measurements were carried out on Mettler-Toledo TGA1, equipped with STARe software. Samples measured in Aluminum pans.

Raman spectra were acquired using RAM-II module connected to Bruker Vertex 70 spectrophotometer.

Melting points were measured using Buchi B-545. Samples were measured inside glass capillaries. The melting points reported herein are the higher temperature limit of the melting range, as measured.

### Example 1

### Preparation of Crystalline Efinaconazole Form A

Efinaconazole (0.5 gr) was dissolved in acetonitrile (1.5 mL) in a round bottom flask. 4.5 ml of a water:ethanol solution in a ratio of 2:1 were added and the mixture maintained at room temperature overnight. Subsequently, the obtained crystalline efinaconazole Form A was filtered.

The X-ray powder diffraction pattern, Raman spectrum, DSC thermogram and TGA thermogram of the obtained crystals are depicted in FIGS. 1-4, respectively.

The measured melting point of the obtained crystals is 86.6° C.

### Example 2

### Preparation of Crystalline Efinaconazole Form B

Crystalline efinaconazole Form B was obtained by the following procedures of Examples 2.1-2.3:

### Example 2.1

Efinaconazole (0.5 gr) was dissolved in diethylether (0.75 mL) in a round bottom flask. Hexane (1.6 ml) was added and the mixture maintained at room temperature overnight. Subsequently, the obtained crystalline efinaconazole Form B was filtered.

The X-ray powder diffraction pattern, Raman spectrum, DSC thermogram and TGA thermogram of the obtained crystals are depicted in FIGS. 5-8, respectively.

The measured melting point of the obtained crystals is 87.5° C.

### Example 2.2

Efinaconazole (1.0 gr) was dissolved in a 1:1 mixture of diisopropylether and hexane (5.0 mL) in a round bottom flask at a temperature of 50° C. The solution was cooled to 5° C. over 1.5 hours, and stirred at 5° C. for an additional 0.5 hour. The obtained crystalline efinaconazole Form B was filtered.

### Example 2.3

Efinaconazole (11.8 gr) was charged into a reactor. Hexane (24 ml) was added and the mixture heated to 60° C. until dissolution was obtained. The solution was cooled to room temperature and stirred for 1 hr. Hexane was added (24 ml). The obtained crystals (Form B, with traces of unknown impurities) were filtered.

### Example 3

### Preparation of Crystalline Efinaconazole Form C

Crystalline efinaconazole Form C was obtained by the following procedures of Examples 3.1-3.2:

### Example 3.1

Efinaconazole (0.5 gr) was charged into a round bottom flask. 2.5 ml of diisopropylether:hexane in a ratio of 1:1 were added and the mixture was stirred at 40° C. until complete dissolution. Subsequently, the solution was cooled to a temperature of 4-5° C. and maintained overnight. The obtained crystalline efinaconazole Form C was then filtered.

The X-ray powder diffraction pattern, Raman spectrum, DSC thermogram and TGA thermogram of the obtained crystals are depicted in FIGS. 9-12, respectively.

The measured melting point of the obtained crystals is 86.2° C.

### Example 3.2

Efinaconazole (10 gr) was charged into a reactor. Cyclohexane (40 ml) was added and the mixture heated to 55° C. until dissolution was obtained. The solution was cooled to 10° C. over 3 hr, and stirred at 10° C. for 0.5 hour. The obtained crystals (Form C, with traces of unknown impurities) were filtered.

### Example 4

### Preparation of Crystalline Efinaconazole p-Toluenesulfonate Form I

Crystalline efinaconazole p-toluenesulfonate Form I was obtained by the following procedures of Examples 8.1-8.2:

### Example 8.1

Efinaconazole (approximately 100 gr) was dissolved in methyltetrahydrofuran (abbreviated herein mTHF) (500 mL) at a temperature of 70° C. A solution of p-toluenesulfonic acid (114 gr) in 2-mTHF (200 mL) was added dropwise; the mixture was then cooled to 20° C. over 2 hours and stirred at 20° C. for another 1 hour. The obtained efinaconazole p-toluenesulfonate crystals were filtered. The X-ray powder diffraction pattern of the obtained crystals is depicted in FIG. 13.

### Example 8.2

Efinaconazole p-toluenesulfonate (8 gr) was suspended in acetone (20 mL) and acetonitrile (20.0) and the mixture stirred at 50° C. for 1 hour. Subsequently, the mixture was cooled to 20° C. over 2 hours. The obtained efinaconazole p-toluenesulfonate crystals were filtered.

1. Crystalline efinaconazole Form A.

2. The crystalline efinaconazole Form A of claim 1, characterized by an X-ray powder diffraction pattern having peaks at 7.6, 10.4, 10.8 and 24.0 degrees 2-theta±0.1 degrees 2-theta.

3. The crystalline efinaconazole Form A of claim 1, characterized by a DSC melting onset at 85.75° C.

4. Crystalline efinaconazole Form B.

5. The crystalline efinaconazole Form B of claim 4, characterized by an X-ray powder diffraction pattern having peaks at 7.7, 10.0, 10.6, 12.5, 23.8 and 34.5 degrees 2-theta±0.1 degrees 2-theta.

6. The crystalline efinaconazole Form B of claim 4, characterized by a DSC melting onset at 85.05° C.

7. Crystalline efinaconazole Form C.

8. The crystalline efinaconazole Form C of claim 7, characterized by an X-ray powder diffraction pattern having peaks at 7.7, 10.1, 11.4, 13.8, 13.9, 17.9, 22.5, 26.2, 26.4 and 29.2 degrees 2-theta±0.1 degrees 2-theta.

9. The crystalline efinaconazole Form C of claim 5, characterized by a DSC melting onset at 86.2° C.

10. (canceled)

11. A pharmaceutical composition comprising crystalline efinaconazole Form A, crystalline efinaconazole Form B, crystalline efinaconazole Form C, or a mixture thereof.
